(19) United States
(12) Patent Application Publication
(10) Pub. No.: US 2024/0270789 A1
(43) Pub. Date: Aug. 15, 2024

(54) METHOD FOR PURIFYING A CATIONIC PROTEIN FRACTION AND FRACTION THUS OBTAINED

(71) Applicant: COMPAGNIE LAITIERE EUROPEENNE, Conde-sur-Vire (FR)

(72) Inventors: Takashi Mikogami, Kawasaki Kanagawa (JP); Carine Lechevin, Saint Gregoire (FR)

(21) Appl. No.: 18/030,972

(22) PCT Filed: Oct. 12, 2021
(86) PCT No.: PCT/EP2021/078094

§ 371 Date: Aug. 24, 2023

(30) Foreign Application Priority Data
Oct. 12, 2020 (FR) FR2010423

(51) Int. Cl. C07K 1/34; C07K 1/36; C07K 14/495; C07K 14/79; C12N 9/08; C12N 9/22

(52) U.S. Cl. CPC C07K 1/34; C07K 1/36; C07K 14/495; C07K 14/79; C12N 9/0065; C12N 9/22; C12Y 111/01007

(57) ABSTRACT

The present invention relates to a method for purifying cationic protein fractions by removal of endotoxins; it also relates to the purified fractions thus obtained.

### CROSS-REFERENCE TO RELATED APPLICATIONS

This application is a National Phase Patent Application and claims priority to and the benefit of International Patent Application No. PCT/EP2021/078094, filed on Oct. 12, 2021, which claims priority to and the benefit of French Patent Application No. FR2010423, filed on Oct. 12, 2020. The entire contents of both of which are incorporated herein by reference.

The present invention relates to a method for purifying cationic protein fractions by removal of endotoxins; it also relates to the purified fractions thus obtained.

The endotoxins are the components of the wall of the Gram-negative bacteria. Released during the lysis or destruction of these bacteria, they are responsible for systemic inflammatory manifestations, such as the septic shock, during infections by this type of bacteria. For this reason, a limit of endotoxin content is defined for the drugs or the injectable products (such as water) by the authorities like the FDA (https://www.fda.gov/inspections-compliance-enforcement-and-criminal-investigations/inspection-technical-guides/bacterial-endotoxinspyrogens). Even outside of injectable uses, the uses such as in the cosmetic field, the medical devices field, and the nutrition field are increasingly requiring more and more a reduction in the endotoxin content of the ingredients used.

The endotoxins, also referred to as lipopolysaccharides (LPS), consist of a lipid (lipid A) with a glycan chain attached. The glycan part is composed of two parts, one part referred to as the core oligosaccharide and the other part referred to as the O side chain polysaccharide (O antigen); FIG. 1 shows their schematic representation (Maeshima & Fernandez 2013). Each LPS molecule has multiple negative charges from the phosphate and acid groups of lipid A and from the core oligosaccharide. The endotoxins are known to be thermally and chemically stable. The endotoxin content is expressed in IU (International Unit) which is equivalent to one EU (Endotoxin Unit) (3.4 Test for bacterial endotoxins, The International Pharmacopoeia—9th edition); as an indication, 1 ng of LPS corresponds to about 10 EU (WHO International Standard, 3rd IS for endotoxin), this may be different depending on the origin of the bacterial strains.

In the scope of the development of drugs based on proteins derived from biotechnological methods and implementing biological material, various methods of protein purification have been developed in order to remove endotoxins; these methods are for example a solvent extraction, the affinity chromatography (such as polymyxin B grafted resins, treatment which is however not authorized for preparing food products), membrane techniques (such as ultrafiltration), ion exchange chromatography, hydrophobic interaction chromatography (Petsch, D., 2000. Endotoxin removal from protein solutions. Journal of Biotechnology 76, 97-119; Ongkudon, C.M., Chew, J.H., Liu, B., Danquah, M.K., 2012. Chromatographic Removal of Endotoxins: A Bioprocess Engineer's Perspective. ISRN Chromatography 2012, 1-9).

However, it is known that the removal of endotoxins from a cationic protein fraction is difficult to achieve because most of the cationic proteins such as lysozyme (Petsch, D., Deckwer, W.-D., Anspach, F.B., 1998. Proteinase K Digestion of Proteins Improves Detection of Bacterial Endotoxins by the LimulusAmebocyte Lysate Assay: Application for Endotoxin Removal from Cationic Proteins. Analytical Biochemistry 259, 42-47), ribonuclease A and lactoferrin (Elass-Rochard, E., Roseanu, A., Legrand, D., Trif, M., Salmon, V., Motas, C., Montreuil, J., Spik, G., 1995. Lactoferrin-lipopolysaccharide interaction: involvement of the 28-34 loop region of human lactoferrin in the high-affinity binding to Escherichia coli 055B5 lipopolysaccharide. Biochem J 312, 839-845) have strong interactions with LPS molecules that have several negative charges.

A method for removing endotoxins bound to a cationic protein, in particular lactoferrin, has been proposed in the WO 2009/009706 (Glanbia Nutritionals); this method comprises the steps of a) binding the protein to a cation exchange resin; b) eluting the endotoxin with a low ionic strength solution in the absence of an added surfactant; c) eluting the protein with a high ionic strength solution. This method allows to obtain a lactoferrin isolate containing less than 1 IU/mg of endotoxins.

A similar method for the removal of endotoxins bound to lactoferrin has been proposed in the WO 2010/112988 (Jean-Paul Perraudin); this method allows to obtain a lactoferrin isolate containing less than 50 pg/mg (i.e. about 0.5 IU/mg) of endotoxin.

These two methods show that from a fraction containing a cationic protein such as the lactoferrin having a high affinity with the cation exchange resins, after fixing this cationic protein on these resins, the endotoxins bound to this cationic protein can be dissociated and removed with a solution of low to medium ionic strength (0.25-0.5 M NaCl solution) without detaching this cationic protein from the cation exchange resins. This affinity with the cation exchange resins is dependent on the positive charges (their magnitude and their location) derived from the constituent cationic amino acids (lysine, arginine and histidine) of the cationic proteins. However, these methods do not allow to remove effectively the endotoxins present or bound to a cationic protein or a fraction of cationic proteins with low to medium affinity with the cation exchange resins, since these proteins would then be eluted together with the endotoxins.

The present invention provides a method allowing to remove efficiently the endotoxins present in a fraction of cationic proteins or proteins bound to a cationic protein or a fraction of cationic proteins regardless of the magnitude and location of their positive charges.

The present invention thus relates to a method for purifying a cationic protein fraction comprising the following steps a) to d):
a) obtaining a solution:
   with a pH of between 6.5 and 7.5;
   comprising cationic proteins all having an isoelectric point greater than 7.5 and acidic proteins having an isoelectric point less than 6.5 in a content less than 1% by weight in relation to the weights of the total proteins;
   with a conductivity greater than 45 mS/cm, preferably greater than 50 mS/cm or 60 mS/cm;
By way of illustration and without limitation, such a solution may be selected from:
   a solution containing the lactoferrin derived from cow's milk eluted with 100 mS/cm of NaCl solution from cation exchange resins (e.g. SP Sepharose Big Beads, Cytiva Life Sciences);
   a solution containing a total fraction of the cationic proteins derived from cow's milk eluted with 10% of NaCl from cation exchange resins (e.g. SPEC 70 SLS, Sartorius);
   a solution containing a total fraction of the cationic proteins derived from cow's milk (containing the lactoperoxidase, the ribonucleases, the lactoferrin) with 40 mS/cm of NaCl solution from cation exchange resins (e.g. SP Sepharose Big Beads, Cytiva Life Sciences), supplemented with the saturated NaCl solution up to the conductivity of 60 mS/cm;
   a concentrated solution of bovine lactoferrin by an ultrafiltration from an eluate at 100 mS/cm;
   a solution of microfiltered whey cationic protein isolate (protein/dry matter >90%), supplemented with the saturated NaCl solution up to the conductivity of 60 mS/cm;
   a powder of the caprine lactoferrin reconstituted with a 5% saline solution;
   a lysozyme powder of egg white reconstituted with a 0.5 M NaCl solution.
b) diafiltering said solution of cationic proteins with endotoxin-free water, preferably ultrafiltered osmosis water, using an ultrafiltration membrane with a cut-off threshold of 5 to 50 kDa, the cut-off threshold value is chosen as a function of molecular weight of cationic proteins of the solution, it is generally between 5 and 50 kDa but may be less than or equal to 20 kDa or between 1 and 20 kDa or between 5 and 20 kDa or 20 kDa, less than or equal to 10 kDa or between 1 and 10 kDa or between 5 and 10 kDa or 10 kDa, less than or equal to 5 kDa or between 1 and 5 kDa or 5 kDa, or 1 kDa, the diafiltration is carried out until a conductivity of less than or equal to 10 mS/cm, preferably less than or equal to 5 mS/cm, and even more preferably less than or equal to 1 mS/cm, is obtained; during this diafiltration, causing the conductivity of the solution of the cationic proteins to drop from greater than 45 mS/cm to less than 10 mS/cm, this solution passes continuously through an anion exchange medium, preferably membrane medium (e.g. Sartobind Q, Sartorus Stedium Biotech) or monolithic medium (e.g. CIMmultus QA, BIA Separations), the medium being such that it has little steric exclusion effect, in order to adsorb and remove substantially all of the endotoxins present in the solution;
c) optionally, microfiltering with a membrane with a cut-off threshold between 0.2 and 1.4 um to reduce the microbial load;
d) optionally, spray drying or freeze drying the solution to obtain a powdered cationic protein isolate.

FIG. 2 shows a schematic representation of the method according to the invention. FIG. 3A and 3B illustrate the example of diagrams allowing the implementation of the step b) of the method according to the invention.

Alternatively, the present invention relates to a method for purifying a cationic protein isolate comprising the following steps a) to d):
a) obtaining a solution:
   with a pH of between 6.5 and 7.5;
   comprising cationic proteins all having an isoelectric point greater than 7.5 and acidic proteins having an isoelectric point less than 6.5 in a content less than 1% by weight in relation to the weights of the total proteins;
   with a conductivity of less than 1 mS/cm;
According to a particular embodiment, this second method is applied to the isolate obtained by the previous method.
b) passing said solution through an anion exchange medium, preferably membrane medium (e.g. Sartobind Q, Sartorus Stedium Biotech) or monolithic medium (e.g. CIMmultus QA, BIA Separtions), the medium being such that it has little steric exclusion effect, in order to adsorb and remove substantially all of the endotoxins present in the solution; the solution is preferably passed through an anion exchange medium several times, preferably at least 3 times;
c) optionally, microfiltering with a membrane having a cut-off threshold between 0.2 and 1.4 um in order to reduce the microbial load to an acceptable level for the conformity of the finished product;
d) optionally, spray drying or freeze drying the solution to obtain a powdered cationic protein isolate.

FIG. 4 illustrates a schematic representation of a device allowing the implementation of the alternative method according to the invention.

The present invention also relates to a cationic protein fraction obtainable or such that it is obtained by the methods according to the invention and such that it has an endotoxin content of less than 5 IU/mg protein, preferably less than 1 IU/mg protein, still more preferably less than 0.1 IU/mg protein.

According to one embodiment, the cationic proteins of the fraction are derived from the milk; they may then consist predominantly of lactoferrin or consist predominantly of lactoperoxidase or consist predominantly of ribonucleases or contain TGF-β in a content greater than 20 μg/g, preferably greater than 50 μg/g, most preferably between 100 and 200 μg/g of protein. Consist predominantly means a fraction comprising at least 50%, but also more than 90% or 95% by weight of protein in relation to the weight of the dry matter. The fractions according to the invention may also contain predominantly a mixture of cationic proteins of the milk or of the whey.

### FIGURES

FIG. 1: Schematic representation of an LPS (Maeshima & Fernandez 2013);

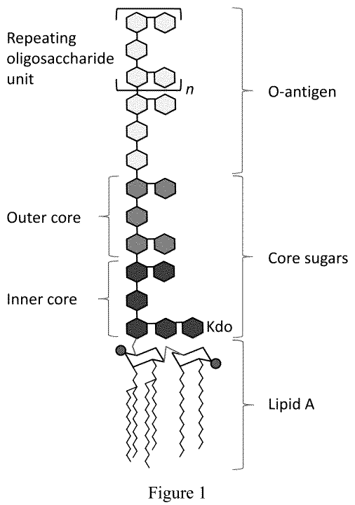

FIG. 2: Schematic representation of the method for purifying the cationic protein isolate;

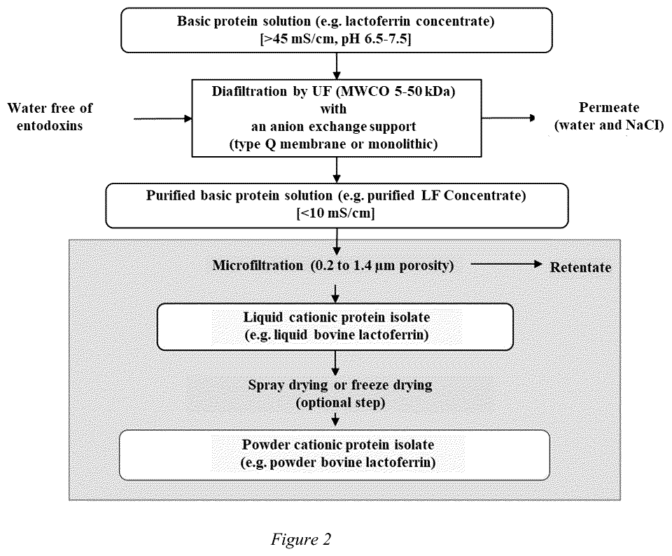

FIG. 3: Examples of diagrams of the method for purifying the cationic protein isolate; A: method combining diafiltration and anion exchange medium in parallel; B: method combining diafiltration and anion exchange medium in series

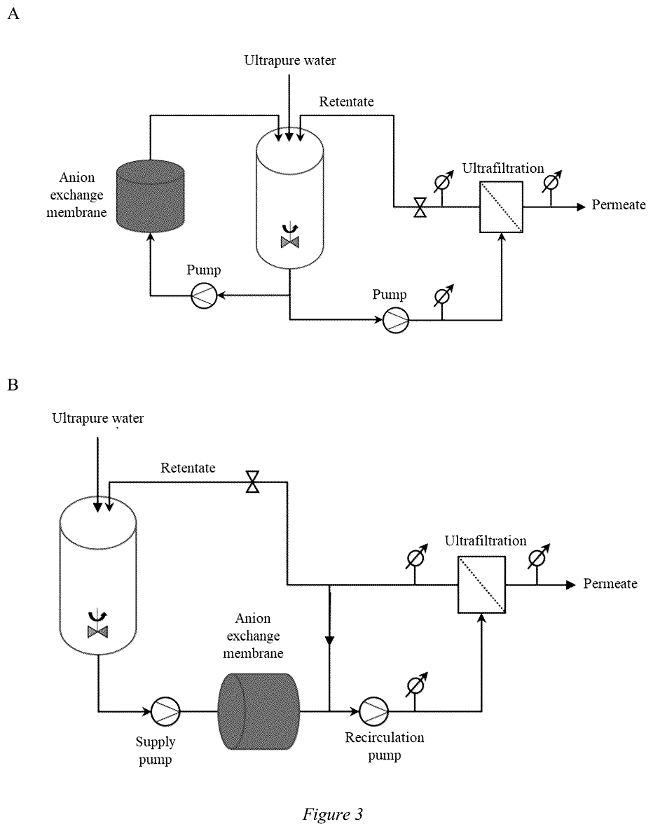

FIG. 4: Schematic representation of the alternative method for purifying the cationic protein isolate.

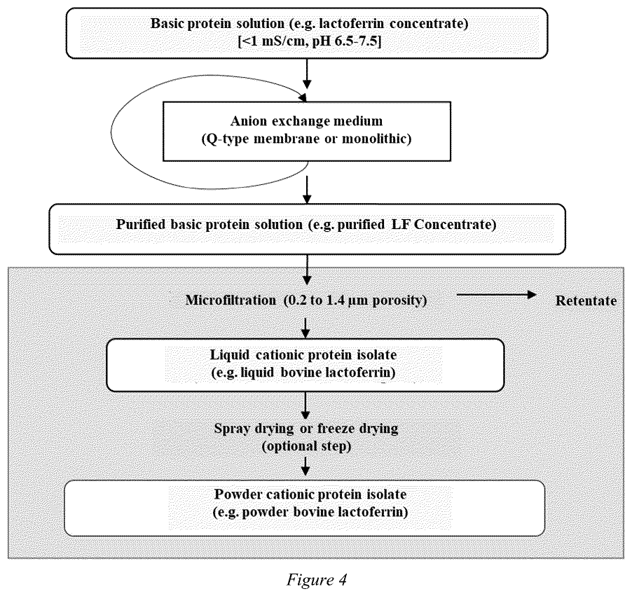

### EXAMPLES

### Example 1: Bench Test with a Liquid Bovine Lactoferrin Concentrate

1) A liquid concentrate of the lactoferrin derived from cow's milk was obtained using an industrial cation exchange chromatography method, SP Sepharose Big Beads (Cytiva Sweden) in a radial flow column (Albert Handtmann Armaturenfabrick GmbH) by passing the pasteurized skimmed cow's milk, then by eluting successively with a NaCl solution at 36 mS/cm and that at 110 mS/cm, finally the 2^{nd} eluate was concentrated on a 20 kDa MWCO ultrafiltration.
   The protein concentration is 13 mg/mL, the purity of bovine lactoferrin on protein is 95%, the conductivity of this solution is 65 mS/cm, and the pH is 6.8 (“Concentrate LF 1”).
2) 75 mL of this liquid bovine lactoferrin concentrate at 65 mS/cm was diafiltered at bench scale using ÄKTA flux s (Cytiva Sweden) with a Start AXH ultrafiltration hollow fiber module (MWCO of 10 kDa). A diafiltration was carried out with ultrapure demineralized water prepared by Milli-Q (Millipore) in a discontinuous way up to 5 mS/cm; when the volume of the retentate reached 50% of the initial volume, ultrapure water was added to the initial volume, which was repeated 5 times. Thus, the demineralized bovine lactoferrin concentrate was obtained (“Concentrate LF 2”).
   The same diafiltration procedure described above was carried out, but this time the concentrate (retentate) was passed in parallel through an anion exchange membrane cartridge of the Q type (quaternary ammonium), Sartobind® Q nano 3 mL (Sartorius Stedim) at a flow rate of 15 mL/min in recirculation for 80 minutes during a gradual decrease in conductivity from 65 to 5 mS/cm by this diafiltration. Thus, the bovine lactoferrin concentrate demineralized by diafiltration and treated with anion exchange membrane was obtained (“concentrate LF 3”).
3) The concentration of endotoxins in each demineralized lactoferrin concentrate obtained by diafiltration was measured by the Lonza's kinetic chromogenic LAL (limulus amoebocyte lysate) assay. In parallel, the concentration of bovine lactoferrin in each concentrate was measured by the HPLC PI method (column C18 300 Å, 0.1% TFA/CH3CN gradient, detection at 280 nm). The results are expressed as IU/mg bovine lactoferrin.

TABLE 1
Endotoxins present in the bovine lactoferrin concentrates

| | Concentrate LF | Concentrate LF | Concentrate LF |
|---|---|---|---|
| | 1 | 2 | 3 |
| Endotoxins | 55.8 | 50.9 | 0.084 |
| (IU/mg LF) | | | |

### Example 2: Bench Test with a Liquid Isolate of Bovine Lactoferrin

1) A liquid microfiltrate of the lactoferrin derived from cow's milk was obtained using an industrial cation exchange chromatography method, SP Sepharose Big Beads (Cytiva Sweden) in a radial flow column (Albert Handtmann Armaturenfabrick GmbH) by passing pasteurized skimmed cow's milk concentrated by reverse osmosis to 130 g/L of dry matter, then eluting successively with a NaCl solution at 38 mS/cm and that a 10%, then the 2nd eluate was concentrated on a 20 kDa MWCO ultrafiltration, then diafiltered on a 10 kDa MWCO ultrafiltration with osmosis water down to 1 mS/cm, finally the diafiltered retentate was microfiltered on a 0.8 μm ceramic membrane in double layer (Membrarox®, Pall Corporation).
2) This liquid bovine lactoferrin isolate was diluted with ultrapure deionized water prepared by Milli-Q (Millipore) to have the protein concentration is 16 mg/mg (w/v) and the purity of bovine lactoferrin on protein is 95%, the conductivity of this solution is 0.15 mS/cm, and the pH is 6.9 (“Isolate LF 1”). 75 mL of this liquid lactoferrin isolate was passed through a Sartobind® Q nano 3 mL cartridge (Sartorius Stedim) at the flow rate of 13 mL/min in recirculation for 90 minutes. (“Isolate LF 2”)
3) The concentration of endotoxins in each lactoferrin isolate was measured by the Lonza's kinetic chromogenic LAL (limulus amoebocyte lysate) assay. In parallel, the concentration of bovine lactoferrin in each concentrate was measured by the HPLC PI method (column C18 300 Å, 0.1% TFA/CH3CN gradient, detection at 280 nm). The results are expressed as IU/mg of bovine lactoferrin.

TABLE 2
Endotoxins present in the bovine lactoferrin isolates

| | Isolate LF 1 | Isolate LF 2 |
|---|---|---|
| Endotoxins (IU/mg LF) | 62.7 | <0.05 |

### Example 3: Bench Test with a Liquid Isolate of Bovine Lactoferrin

1) A liquid microfiltrate of the lactoferrin derived from cow's milk was obtained using an industrial cation exchange chromatography method, SP Sepharose Big Beads (Cytiva Sweden) in a radial flow column (Albert Handtmann Armaturenfabrick GmbH) by passing pasteurized skimmed cow's milk, then eluting successively with a NaCl solution at 36 mS/cm and that at 110 mS/cm, then the 2nd eluate was concentrated on a 20 kDa MWCO ultrafiltration, then diafiltered on a 10 kDa MWCO ultrafiltration with osmosis water up to 1 mS/cm, finally the diafiltered retentate was microfiltered on a 1.4 μm ceramic membrane in double layer (Membrarox®, Pall Corporation).
   The protein concentration is 147 mg/mL, the purity of bovine lactoferrin on protein is 95%, the conductivity of this solution is 0.8 mS/cm, and the pH is 6.8 (“Isolate LF 3”).
2) 70 mL of this liquid lactoferrin isolate was passed through a Sartobind® Q nano 3 mL cartridge (Sartorius Stedim) at a flow rate of 6 mL/min (“Isolate LF 4”).
   The recovered lactoferrin liquid isolate (65 mL) was passed through the Sartobind® Q nano 3 mL cartridge, and then the recovered lactoferrin liquid isolate was again 1 passed through the Sartobind® Q nano 3 mL cartridge, a total of 3 passages (“Isolate LF 5”).
   The recovered lactoferrin liquid isolate (60 mL) was passed through the Sartobind® Q nano 3 mL cartridge in recirculation for 30 minutes at a flow rate of 6 mL/min in a total of 6 equivalent passages (“Isolate LF 6”).
   The recovered lactoferrin liquid isolate (55 mL) was passed through the Sartobind® Q nano 3 mL cartridge in recirculation for 37 minutes at a flow rate of 6 mL/min in a total of 10 equivalent passages (“Isolate LF 7”).
3) The concentration of endotoxins in each lactoferrin isolate was measured by the Lonza's kinetic chromogenic LAL (limulus amoebocyte lysate) assay. Results are expressed as IU/mg of proteins.

TABLE 3
Endotoxins present in the bovine lactoferrin isolates

| | Isolate LF | Isolate LF | Isolate LF | Isolate LF | Isolate LF |
|---|---|---|---|---|---|
| | 3 | 4 | 5 | 6 | 7 |
| Endotoxins | 59.8 | 7.2 | 4.6 | 2.2 | 1.5 |
| (IU/mg of | | | | | |
| proteins) | | | | | |

### Example 4: Bench Test with a Liquid Isolate of TGF-β Containing Cationic Proteins Derived From Cow'S Milk

1) A TGF-β-containing cationic protein fraction derived from cow's milk was obtained according to the method described in the Example 1 of the Patent EP 1912513. The TGF-β2 content analyzed by the ELISA kit (Quntikine TGF-2, R&D Systems) in the microfiltrate obtained before the spray drying was 115 μg/g of proteins.
2) This liquid cationic protein isolate containing TGF-β was diluted with ultrapure deionized water prepared by Milli-Q (Millipore) to have a protein concentration of 2.6 mg/mg (w/v), the conductivity of this solution is 0.89 mS/cm, and the pH is 7.1 (“Milk cationic protein isolate 1”). 50 mL of this liquid milk cationic protein isolate was recirculated through a Sartobind® Q nano 3 mL cartridge (Sartorius Stedim) at a flow rate of 5 mL/min for 70 minutes. (“Milk cationic protein isolate 2”)
3) The concentration of endotoxins in each liquid milk cationic protein isolate was measured by the Lonza's kinetic chromogenic LAL (limulus amoebocyte lysate) assay. Results are expressed as IU/mg of proteins.

TABLE 4
Endotoxins present in the milk cationic
protein isolates containing TGF-β

| | Milk cationic protein | Milk cationic protein |
|---|---|---|
| | isolate 1 | isolate 2 |
| Endotoxins | 9.75 | 0.099 |
| (IU/mg of proteins) | | |

1. A method for purifying a cationic protein fraction comprising the following steps:
a) obtaining a solution:
with a pH of between 6.5 and 7.5;
comprising cationic proteins all having an isoelectric point greater than 7.5 and acidic proteins having an isoelectric point less than 6.5 in a content less than 1% by weight in relation to a total weight of all proteins;
with a conductivity greater than 45 mS/cm;
b) diafiltering said solution with endotoxin-free water using an ultrafiltration membrane with a cut-off threshold of 5 to 50 kDa, until a conductivity of less than or equal to 10 mS/cm is obtained; during the diafiltration, the solution passes continuously through an anion exchange medium;
c) optionally, microfiltering with a membrane with a cut-off threshold between 0.2 and 1.4 μm;
d) optionally, spray drying or freeze drying the solution.

2. A method for purifying a cationic protein isolate comprising the following steps:
a) obtaining a solution:
with a pH of between 6.5 and 7.5;
comprising cationic proteins all having an isoelectric point greater than 7.5 and acidic proteins having an isoelectric point less than 6.5 in a content less than 1% by weight in relation to a total weight of all proteins;
with a conductivity of less than 1 mS/cm;
b) passing said solution through an anion exchange medium;
c) optionally, microfiltering with a membrane having a cut-off threshold between 0.2 and 1.4 μm;
d) optionally, spray drying or freeze drying the solution to obtain a powdered cationic protein isolate.

3. A cationic protein fraction obtained by the method according to claim 1, characterized in that it has an endotoxin content of less than 0.1 IU/mg of protein.

4. A cationic protein fraction obtained by the method according to claim 1, characterized in that it has an endotoxin content of less than 5 IU/mg of protein.

5. The cationic protein fraction according to claim 3, characterized in that the cationic proteins of the fraction are derived from milk.

6. The cationic protein fraction according to claim 3, characterized in that a majority of the cationic proteins are lactoferrin.

7. The cationic protein fraction according to claim 3, characterized in that a majority of the cationic proteins are lactoperoxidase.

8. The cationic protein fraction according to claim 3, characterized in that a majority of the cationic proteins are ribonucleases.

9. The cationic protein fraction according to claim 3, characterized in that the cationic proteins contain TGF-β in an amount of greater than 20 μg/g of protein.

10. The method of claim 1, wherein the diafiltration comprises using the using the ultrafiltration membrane until the conductivity reaches less than or equal to 5 mS/cm.

11. The method of claim 2, wherein the passing the solution through the anion exchange medium comprises passing the solution through the anion exchange medium at least 3 times.

12. A cationic protein fraction obtained by the method according to claim 2, characterized in that it has an endotoxin content of less than 0.1 IU/mg of protein.

13. A cationic protein fraction obtained by the method according to claim 2, characterized in that it has an endotoxin content of less than 5 IU/mg of protein.

14. The cationic protein fraction according to claim 4, characterized in that the cationic proteins of the fraction are derived from milk.

15. The cationic protein fraction according to claim 4, characterized in that a majority of the cationic proteins are lactoferrin.

16. The cationic protein fraction according to claim 4, characterized in that a majority of the cationic proteins are lactoperoxidase.

17. The cationic protein fraction according to claim 4, characterized in that a majority of the cationic proteins are ribonucleases.

18. The cationic protein fraction according to claim 4, characterized in that the cationic proteins contain TGF-β in an amount of greater than 20 μg/g of protein.
